(19) United States
(12) Patent Application Publication
(10) Pub. No.: US 2019/0110960 A1
(43) Pub. Date: Apr. 18, 2019

(54) HARDENABLE DENTAL IMPRESSION COMPOSITION COMPRISING A POLYMERIC FILLER PARTICLES AND USE THEREOF

(71) Applicant: 3M INNOVATIVE PROPERTIES COMPANY, St. Paul, MN (US)

(72) Inventors: Andreas R. Maurer, Langenneufnach (DE); Peter Bissinger, Diessen (DE); Christoph Schulte, Windach (DE); Arne Thaler, Emmerting (DE); Joachim W. Zech, Kaufering (DE); Johann Fetz, Windach (DE); Peter U. Osswald, Turkheim (DE); Johannes M. Leykauff, Unterhausen (DE)

(21) Appl. No.: 15/533,097

(22) PCT Filed: Dec. 3, 2015
(86) PCT No.: PCT/US2015/063580

§ 371 Date: Jun. 5, 2017

(30) Foreign Application Priority Data
Dec. 16, 2014 (EP) 14198124.1

(51) Int. Cl. A61K 6/10; A61K 6/00; A61C 9/00

(52) U.S. Cl. CPC A61K 6/10; A61K 6/0088; A61K 6/0011; A61K 6/0008; A61C 9/00

(57) ABSTRACT

The present invention relates to a hardenable dental composition comprising (A) a curable component comprising curable moieties (AC), (B) a crosslinker component as component (B) capable of crosslinking said component (A), (C) a catalyst as component (C) capable of catalyzing a crosslinking reaction of component (A) and component (B), (D) polymeric particles as filler component (D), the polymeric particles having a maximum particle size of 150 μm or below and being composed of organic polymers, silicone elastomers or a combination or mixture thereof and preferably (E1) an inorganic filler component having a BET surface from 50 to 400 m²/g.

The invention also relates to the use of a polymeric particulate filler component (D) for producing a hardenable dental composition, the hardenable dental composition being in particular useful as dental impression or dental retraction material or for dental impression or dental retraction purposes.

### FIELD OF INVENTION

The invention is directed to a hardenable dental composition comprising polymeric particles as filler. The hardenable dental composition is particularly useful in the dental field, e.g. as dental impression material.

### BACKGROUND

Producing dental replacement parts like crowns and bridges requires the exact determination of the dental situation in the mouth of the patient. Otherwise, the dental replacement parts will not accurately fit.

For determining the dental situation in the mouth of a patient different methods are know. Besides imaging and computer based methods, a huge portion of this task is still accomplished by using conventional dental impression materials.

Dental impression materials can be classified according to their curing mechanism (e.g. addition curing or condensation curing). Dental impression materials can also be classified according to their consistency. Besides low viscous dental impression materials, there exists highly viscous, so-called putty like dental impression materials.

Dental impression materials are typically provided as two component systems which consist of a base and a catalyst paste and which are mixed before use.

Due to its high viscosity, mixing putty like dental impression materials with an automated system like the Pentamix™ device (3M ESPE) can be difficult. Thus, often putty like dental impression materials are mixed (i.e. kneaded) by hand.

The materials are typically pasty and cure in the mouth of the patient due to a chemical curing mechanism. The cured material is removed from the mouth of the patient and the obtained impression represents a negative image of the dental situation. The impression is then typically filled with plaster to obtain a positive model. The positive model is used for designing the desired dental replacement part.

For an easy removal of the material from the mouth of a patient, the cured dental impression material should not be too hard.

A reduced hardness can be achieved by e.g. reducing the filler content of the material. This, however, often causes the material to become thinner from a viscosity standpoint of view.

A reduced hardness can also be achieved by adding high viscous silicone oil. This, however, may cause the material to become more tacky and difficult to be mixed in particular by hand.

A reduction of the stickiness of the material can be achieved by addition of paraffin or mineral oil. The paraffin or mineral oil migrates to the surface of the composition and forms a kind of separation layer on the surface of the pastes to be mixed.

However, a disadvantage of using paraffin or mineral oil is the fact that these components often tend to separate from the pastes giving them a greasy feeling.

U.S. Pat. No. 7,186,758 B2 (Zech et al.) describes a two-component addition-cured silicone impression compound comprising between 4 to 10 wt.-% of at least one liquid paraffin or at least one white mineral oil and having a consistency stipulated by ISO 4823 of less than or equal to 35 mm.

U.S. Pat. No. 4,891,400 (Schwabe et al.) relates a dental molding composition comprising an organopolysiloxane with reactive end groups, a catalyst, a filler and an iso-paraffine having 8 to 24 carbon atoms.

U.S. Pat. No. 4,600,731 (Louis et al.) describes dental impression composition containing (a) a platinum compound or complex; (b) a diorganopolysiloxane having at least 2 SiC-bonded vinyl groups per molecule; (c) a hydrocarbon which is liquid or spreadable at room temperature and is free of aliphatic unsaturated carbon-carbon bonds; (d) a hydrophobic silicon dioxide having a surface area of at least 50 m2/g and other additives, if desired, such as pigments, fragrances and agents which control the rate of the platinum catalyzed addition of the Si-bonded hydrogen to SiC-bonded vinyl groups.

U.S. Pat. No. 6,552,104 B1 (Hare) refers to very high viscosity (putty) two component polymerizable polyorganosiloxane compositions for use in making non-sweating, low liquid mass loss dental impressions having improved tear strength, handling and wettability. It is said that the handling is improved by the addition of a very high viscosity linear vinyl terminated polydimethylsiloxane which reduces tackiness and improves the shelf stability.

None of the described methods and materials is fully satisfactory. There is still a desire for an improved dental composition.

### DESCRIPTION OF INVENTION

It is a general object to provide an improved dental material which addresses one or more of the issues mentioned in the present text.

From a patient's standpoint of view, there is a need for a dental material which can be removed from the patient's dentition more easily.

Ideally, the dental material should have a sufficiently high consistency but a reduced hardness after hardening, ideally combined with a good elongation at break value.

From a practitioner's standpoint of view, the dental material should also be easily mixable by hand without showing oil separation and have the desired properties once hardened.

It was found that one or more of the above objects can be achieved with the hardenable composition described in the present text.

According to one aspect, the invention is directed to a two-component hardenable dental composition, the dental composition comprising

(A) curable component (A) comprising curable moieties (AC),

(B) crosslinker component as component (B) capable of crosslinking said component (A),

(C) catalyst as component (C) capable of catalyzing a crosslinking reaction of component (A) and component (B),

(D) polymeric particles as filler component (D), the polymeric particles having a maximum particle size of 150 μm or below and being composed of organic polymers, silicone elastomers or a mixture thereof, filler component (D) being preferably present in an amount from 2 to 70 wt.-% with respect to the weight of the whole composition and preferably

(E1) an inorganic filler component having a BET surface from 50 to 400 m²/g.

A further embodiment of the invention is directed to a process of taking an impression of the dental situation in the mouth of a patient or conducting a retraction, the process comprising the steps of:

providing the hardenable composition as described in the present text,

bringing the hardenable composition in contact with the surface of the dental situation in the mouth of a patient,

letting the hardenable composition harden,

removing the hardened composition from the mouth of the patient.

The invention is also directed to the use of polymeric particles as described in the present text for producing a dental impression material or dental retraction material.

The invention is also directed to the use of the polymeric particulate filler component as described in the present text for lowering the Shore hardness and increasing the elongation at break value of the hardened dental composition while maintaining the consistency of the hardenable dental composition described in the present text.

### FIGURES

FIG. 1 is a diagram exemplifying schematically the dependency of Shore hardness with respect to filler content for two different kinds of fillers—inorganic and polymeric organic fillers.

Unless defined differently, for this description the following terms shall have the given meaning:

A “dental composition” or a “composition for dental use” or a “composition to be used in the dental field” is any composition which can be used in the dental field. In this respect the composition should not be detrimental to the patients' health and thus free of hazardous and toxic components being able to migrate out of the composition. Dental compositions are typically hardenable compositions, which can be hardened at ambient conditions, including a temperature range from about 15 to 50° C. or from about 20 to 40° C. within a time frame of about 30 min or 20 min or 10 min. Higher temperatures are not recommended as they might cause pain to the patient and may be detrimental to the patient's health. Dental compositions are typically provided to the practitioner in comparable small volumes, that is volumes in the range from about 0.1 to about 500 ml or from about 0.5 to about 100 ml or from about 1 to about 50 ml. Thus, the storage volume of useful packaging devices is within these ranges.

A “dental impression material” is a material used for making impressions of the tooth structure including the gingiva. A dental impression material is usually applied on a dental impression tray. A dental impression material can be based on different chemical substances and crosslink by various chemical reactions (including addition curing and condensation curing materials). Typical examples include silicone based impression materials (e.g. VPS materials) and polyether based impression materials and mixtures of those.

A “putty like dental impression material” is a kneadable dental impression material having a consistency of 35 mm or below if determined according to ISO 4823.

A “dental retraction material” is a material intended to be placed in the gingival sulcus, that is, the natural space between the hard dental tissue (i.e. tooth structure) and the gum tissue that surrounds the hard dental tissue. Once placed in the gingival sulcus, the dental retraction material will exert pressure on the surrounding tissue resulting in a widening of the gingival sulcus to enable the practitioner to get a more precise impression of the dental situation below the gum line during a dental impression process. Like a dental impression material, a dental retraction material is removed from the mouth of the patient after use.

The term “compound” or “component” is a chemical substance which has a particular molecular identity or is made of a mixture of such substances, e.g., polymeric substances.

A “liquid” is any solvent or liquid which is able to at least partially disperse, dissolve or suspend the components being present in the inventive composition at ambient conditions (e.g. 23° C.).

By “paste” is meant a soft, viscous mass of solids (i.e. particles) dispersed in a liquid.

A “particle” or “particulate filler” means a substance being a solid having a shape which can be geometrically determined. The shape can be regular or irregular. Particles can be analysed with respect to e.g. grain size and grain size distribution. A particulate filler is composed of free-flowing particles. “Free-flowing” means that the particulate filler can be sieved, that is, it behaves like e.g. dry powdered sugar.

“Particle size distribution and maximum particle size” includes the size of agglomerated or aggregated particles. The maximum particle size can be determined by laser diffraction using a Cilas 1064 Granulometer in “Dry Mode”. Results are calculated using the Fraunhofer approximation without Mie correction.

“Polymeric particle” means the particles have been produced by polymerization (e.g. free-radical polymerization) of monomers comprising polymerizable moieties (e.g. unsaturated moieties). Particles obtained by sol-gel condensation are not considered polymeric filler particles. Polymeric particles are often granular or spherically shaped.

Polymeric filler particle(s) include organic polymeric material(s) and/or silicone elastomeric material(s), especially silicone elastomers comprising dimethylsiloxane moieties.

An “organic polymer” is a macromolecule composed of repeated subunits or moieties. Organic polymers are further characterized by a molecular weight distribution. In contrast to inorganic substances, organic polymers only comprise, contain or consist of the organic elements C, H, N, O, P, S, F, Cl, Br, I as main elements (i.e. more than 50%).

“Elastomeric” means rubber-elastic or rubber-like. Elastomeric materials can be characterized e.g. by a certain tensile strength and/or elongation at break. Other means for characterizing elastomeric materials include the measurement e.g. of the Young's modulus. Elastomeric materials typically have an E-modulus in the range from 0.8 to 10 MPa or from 1 to 8 MPa or from 1.5 to 6 MPa (determined e.g. according to DIN 53504, thickness of sample: 2 mm).

A “silicone elastomer” is an elastomeric polymer comprising silicone units, i.e. comprising the elements Si, O, C and H, in particular dimethylsiloxane (—O—Si(CH₃)₂—) units.

The term “silicone,” as used herein, refers to a polymer having, for the most part, alternating silicon and oxygen atoms (i.e., a polysiloxane chemical structure) and having sufficient pendant functional groups to undergo a setting reaction in the presence of a crosslinker compound and a catalyst compound.

The terms “vulcanizing, hardening, crosslinking, setting, curing” are used interchangeable and refer to silicones that have as a common attribute the development of a crosslinked elastomer from relatively low molecular weight linear or branched polymers by means of a chemical reaction that simultaneously forms these crosslinks and effectively extends chain length at room temperature.

“(Meth)acryl” means “acryl” and “methacryl”.

“Urethane” means a moiety with the structural element “—O—CO—NH—”.

“Poly” means that the respective substance contains at least 10 repeating units of a certain monomer moiety.

The term “hydrosilation” means the addition of a compound comprising SiH-groups to a compound containing an aliphatic multiple bond (e.g., an olefinic or acetylenic unsaturation), preferably a vinyl group, —CH═CH₂.

“Room temperature hardening or curing” implies that the curing reaction can proceed and completed at temperatures at or near 25° C. For example, the oral cavity of the mouth has an average temperature of approximately 32° C. and is therefore near room temperature. Certain “high” temperature cured materials are designed to cure only at relatively high temperatures (e.g., >50° C. or >100° C.) and are stable (i.e., the curing reaction is inhibited) at room temperature.

“Non-swellable hardenable composition” means that the composition does not expand during the curing or hardening process by more than 10 or 5 or 1% by volume compared with the initial volume.

“Non-swellable polymeric particles” means that the particles do not show a volume expansion of more than 5 or 1% by volume within 5 min, if dispersed in water.

The term “crosslinked polymer” refers to polymers that have reacted with the functional group or groups of the polymer chains to lengthen them and connect them, e.g., to form a crosslinked network characteristic of a silicone elastomer. In contrast to a thermoplastic polymer (i.e., a polymer that softens and flows upon heating) a crosslinked polymer, after crosslinking, is characteristically incapable of further flow.

The term “working time” as used herein, refers to the time between the initiation of the setting reaction (e.g., when a vinyl-containing organopolysiloxane, a organohydropolysiloxane, and a platinum catalyst are mixed) and the time the setting reaction has proceeded to the point at which it is no longer practical to perform further physical work upon the system, e.g., reform it, for its intended purpose. When the reaction has proceeded to this later point the material is said to have reached its “gel point.” The working time preferably provides enough time to mix and place the composition into its desired form. For many dental impression compositions and applications the working time under conditions of use can be greater than about 30 s (seconds), or greater than about 1 min (minute), or greater than about 2 min. Thus, the working time is typically within a range of about 30 s to about 3 min or about 1 min to about 2 min. So-called “fast-setting” compositions typically have a shorter working time, e.g. less than about 2 min or less than about 1.5 min.

The terms “set time” or “setting time” as used herein, refer to the time at which sufficient curing has occurred so that essentially the material's final cured-state properties are obtained. For a silicone impression material the set time is that time at which one may remove the material from the surface being replicated without causing permanent deformation of the silicone material. The setting time may be approximated, for example, by measuring the torque of the reacting composition on an oscillatory rheometer. When the torque value reaches a maximum value the material is said to be fully set. An arbitrary torque value which is less than the typical maximum value (e.g. 90% of the maximum value) may alternatively be used as a practical approximation of the set time. In general, shorter setting times are preferred over longer setting times. For dental impression compositions the setting time occurs at a time preferably less than about 10 minutes after initiation of the reaction. More preferably the setting time is less than the sum of about 5 minutes plus the working time.

More specifically, the setting time is the time between positioning of the spoon with the dental material in the mouth of the patient and removal of the cured material, and can also be called the mouth residence time or period. Setting times of <about 5 min mouth residence time, preferably <about 4 min, and particularly preferably <about 2 min are desirable properties for the dentist working with impression materials.

For example, the one-phase impression material Imprint™ (3M ESPE) has a setting time of about 5 min, while a typical alginate impression material such as Palgat™ (3M ESPE) has a setting time of about 4 min.

The term “molecular weight” refers to the number average of the molecular weight, as is conventionally determined for the individual classes of polymers by gel permeation chromatography (GPC) against a standard of defined molecular weight. Suitable measurement methods will be known to the person skilled in the art.

Furthermore, the determination of the molecular weights and the molecular weight distribution of polymeric polyols can be carried out, for example, by means of end group determination, for example by nuclear magnetic resonance (NMR) methods. Also suitable for the determination of the molecular weights and the molecular weight distribution of polymeric polyols is the determination of the hydroxyl value.

“Hydrophilating agents” are agents that are able to either lower the surface tension of water, if used alone (like surfactants), or contribute to a lower surface tension, if used in combination with a surfactant (sometimes referred to as wetting-enabler). If desired, the effect of lowering the surface tension of water can be measured by determining the water-contact angle as described in more detail below.

The term “automixer-suitable material” relates to a multi-component material which can be dispensed, for example, from a two-component disposable cartridge through a static mixer, e.g., of SulzerMixpac Company (cf. U.S. Pat. No. 5,464,131, EP 0 730 913 A1) or from film bags in dual-chamber reusable cartridges through a dynamic mixer, e.g., in the “Pentamix™”, “Pentamix™ 2” and “Pentamix™ 3” devices of 3M ESPE Company (cf. U.S. Pat. Nos. 5,286,105 and 5,249,862).

“Ambient conditions” mean the conditions which the inventive composition is usually subjected to during storage and handling. Ambient conditions may, for example, be a pressure of about 900 to about 1100 mbar, a temperature of about −10 to about 60° C. and a relative humidity of about 10 to about 100%. In the laboratory ambient conditions are adjusted to about 23° C. and about 1013 mbar. In the dental and orthodontic field ambient conditions are reasonably understood as a pressure of about 950 to about 1050 mbar, temperature of about 15 to about 40° C. and relative humidity of about 20 to about 80%.

As used herein, “a”, “an”, “the”, “at least one” and “one or more” are used interchangeably. The terms “comprises” or “contains” and variations thereof do not have a limiting meaning where these terms appear in the description and claims. The term “comprising” also includes the more limited expressions “consisting essentially of” and “consisting of”.

Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g. the term “additive(s)” means one additive and more additives (e.g. 2, 3, 4, etc.).

“Comprise” includes the terms “contain”, “essentially consists of” and “consists of”. Unless otherwise indicated, all numbers expressing quantities of ingredients, measurement of physical properties such as described below and so forth used in the specification and claims are to be understood as being modified in all instances by the term “about.”

The invention is advantageous in a variety of aspects. It was found that by replacing inorganic filler particles of a given formulation by polymeric filler particles, the hardness of the cured composition can be reduced, although the consistencies of the pastes to be mixed for obtaining the curable composition remain essentially unaffected before mixing.

The present invention also enables the formulation of a composition having a high filler load and thus a high consistency without negatively affecting the Shore hardness.

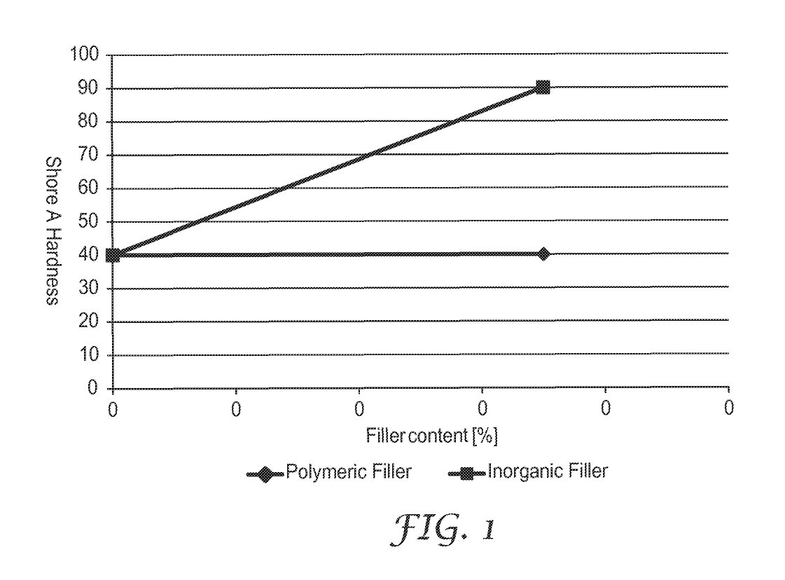

This is schematically shown in FIG. 1. If an inorganic filler was used, the Shore hardness of the cured composition increased with higher filler load. In contrast, if a polymeric filler was used, the Shore hardness of the cured composition can either be lowered, if the filler content is kept constant or essentially kept at the same level, if the filler load is increased.

It was also found that by adding polymeric particles to the curable composition, the tendency of paraffin or mineral oil (if present) to migrate out of the composition is decreased and the pastes are essentially non-sticky to gloves which may be used during mixing the composition. This facilitates mixing of the pastes by hand but at the same time makes it easier to remove the cured composition from the patient's mouth.

In addition, it was found that the cured composition can still be cut and trimmed easily with a scalpel, if desired, which can be important for dentists applying certain dental impression techniques.

Thus, the present invention allows the provision of a dental composition which can easily be mixed by hand (e.g. is non-sticky during mixing but still has a sufficient consistency) and which can easily be removed from the mouth of a patient after curing (due to its reduced hardness).

Such a material can also be used for dental retraction purposes as it has a sufficient consistency before hardening for placing it into the sulcus of a tooth and exerting pressure on the surrounding soft tissue, thus enabling a widening of the sulcus.

The hardenable dental composition described in the present text is a room temperature curable composition. That is, it a composition showing a Shore hardness A of at least 20 within 15 min after mixing of the components of the composition at 23° C.

Before hardening means, that the hardenable composition is analyzed with respect to its properties immediately after mixing of the respective components or parts.

The hardenable composition described in the present text enables the practitioner also to formulate a putty-like material. High consistency (i.e. low value in mm) means that the composition shows a reduced flowing behaviour under load.

The consistency which can be achieved is typically with the range commercial impression materials have. These consistencies are usually classified as follows (ISO 4823):

consistency 3: corresponding to at least 36 mm;

consistency 2: corresponding to 31 to 41 mm;

consistency 1: corresponding to utmost 35 mm;

consistency 0: corresponding to utmost 35 mm.

The hardenable composition described in the present text can also be characterized by at least one or more or all of the following parameters after hardening:

Shore A hardness (according to DIN 53505; 24 h): at least 20 or at least 30 or at least 35;

Elongation at break (according to DIN 53504): at least 20%, or at least 50%, or at least 100%, or at least 200%;

Recovery from deformation (according to ISO 4823): at least 90%, or at least 95%, or at least 98%;

Tensile strength (according to DIN 53504): at least 0.2 or at least 2.0 or at least 3.0 MPa;

Curing time: Shore hardness A of at least 20, or at least 25, or at least 30 within 15 min at 23° C.;

Water contact angle: less than 20° or less than 15° at a water drop age of 10 s, 60 s after mixing of the components.

If desired, the parameters can be determined as described in the Example section.

The hardenable composition described in the present text comprises a curable component as component (A). The curable component (A) comprises curable moieties (AC).

Hardening of component(s) (A) comprising curable moieties (AC) can be achieved by different mechanisms.

According to one embodiment, hardening of the curable composition is effected by a polyaddition reaction.

This curing mechanism is typically based upon the polyaddition of silanes with olefinically unsaturated double bonds (e.g. vinyl groups) in the presence of a catalyst, such as Pt containing compound. The respective compositions are often referred to as VPS materials and the curing mechanism as hydrosilation.

According to this embodiment, the invention features a composition comprising as components (A) at least one organopolysiloxane with at least 2 aliphatically unsaturated groups.

The organopolysiloxane is a molecule in which at least two organic groups are groups with an ethylenically unsaturated double bond. Generally, the groups with an ethylenically unsaturated double bond can be located on any monomeric unit of the organopolysiloxane. It can, however, be preferred, that the groups with an ethylenically unsaturated double bond are located on or at least near the terminal monomeric units of the polymer chain of the organopolysiloxane. In another embodiment, at least two of the groups with an ethylenically unsaturated double bond are located on the terminal monomeric units of the polymer chain.

The term “monomeric units” as used throughout the present text relates to repeating structural elements in the polymer that form the polymer backbone, unless expressly stated otherwise.

Preferred organopolysiloxanes of this general structure are represented by the following formula (I):

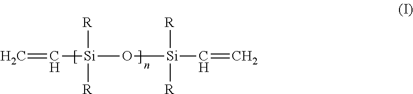

in which the radicals R, independently from each other, represent a non-substituted or substituted, monovalent hydrocarbon group with 1 to 6 C atoms, which is preferably free from aliphatic multiple bonds and where n generally can be chosen such that the viscosity of the organopolysiloxanes lies between 4 and 1,000,000 mPas or between 6 and 500,000 or between 10 and 100,000 mPas. The parameter n can, e.g., be in the range of 10 to 10,000.

Generally, the radicals R in the above formula can represent any non-substituted or substituted, monovalent hydrocarbon group with 1 to about 6 C atoms. Non-substituted or substituted, monovalent hydrocarbon groups with 1 to about 6 C atoms can be linear or, if the number of carbon atoms exceeds 2, branched or cyclic. Generally, the radicals R can be equipped with any type of substituent or substituents provided they do not interfere with any other constituents or substituents of the composition and do not interfere with the curing reaction.

The term “interfere” as used in the context of the present text relates to any influence of such a substituent on at least one of the other substituents or constituents of the composition or the curing reaction, or both, which might be detrimental to the properties of the hardened product.

The term “detrimental” as used in the context of the present text relates to a change of properties of the precursors or the cured product that negatively affect the usefulness of the precursors or the cured product in their intended use.

In another embodiment of the invention, at least about 50% of the radicals R are methyl groups. Examples of other radicals R that can be present in the organopolysiloxanes according to the above formula are ethyl, propyl, isopropyl, n-butyl, tert.butyl, the pentyl isomers, the hexyl isomers, vinyl, propenyl, isopropenyl, 2- and 3-n-butenyl, the pentenyl isomers, the hexenyl isomers, fluorine substituted aliphatic radicals like 3,3,3-trifluoropropyl groups, cyclopentyl or cyclohexyl groups, cyclopentenyl or cyclohexenyl groups or aromatic or heteroaromatic groups like phenyl or substituted phenyl groups. Examples for such molecules are described in U.S. Pat. No. 4,035,453, the disclosure of which, especially regarding the above mentioned molecules, their chemical constitution and their preparation, is included herein by reference.

The preparation of molecules according to the above-mentioned formula would generally be understood by the skilled person based upon the teachings of the prior art regarding similar molecules.

Particularly preferred are linear polydimethylsiloxanes according to the above formula having viscosities within the specified viscosity ranges and end groups comprising dimethylvinylsiloxy units and methyl groups as the radicals R.

A component (A) which can be employed can consist of one type (A1) of organopolysiloxane. The organopolysiloxane can have a viscosity starting in the range of 5 to 1,000,000 mPas, or 10 to 500,000 mPas or 20 to 50,000 or 30 to 40,000 mPas.

It is, however, also possible that component (A) comprises two or more constituents, (A1), (A2) and so on, which can differ, e.g., in the chemical composition of their backbone, or their molecular weight, or their substituents or their viscosity, or any other differentiating feature or two or more of the above mentioned features.

In one embodiment the difference in viscosities of different constituents of component (A) can be higher than a factor of 2, e.g., higher than a factor of 5, higher than a factor of 10, higher than a factor of 20, higher than a factor of 30, higher than a factor of 40, higher than a factor of 50, higher than a factor of 60, higher than a factor of 70, higher than a factor of 80, higher than a factor of 90 or higher than a factor of 100. The difference in viscosities can be even higher, e.g., higher than a factor of 200, higher than a factor of 300, higher than a factor of 500, higher than a factor of 800, higher than a factor of 1,000 or higher than a factor of 5,000, it should, however, preferably not exceed a value higher than a factor of 10,000. It should be kept in mind that the values mentioned above relate to a factor for the difference in viscosities, not the viscosity values themselves.

If component (A) contains constituents of different viscosities, the ratio of the amount of constituent with the lowest viscosity to the amount of constituent with the highest viscosity can be chosen relatively freely, depending on the desired properties of the precursors and the cured resin. It can, however, be advantageous when the ratio of the amount of constituent with the lowest viscosity to the amount of constituent with the highest viscosity is within a range of from 1:20 to 20:1, especially 1:10 to 10:1 or 1:5 to 5:1. Good results can e.g. be obtained with ratios of from 1:3 to 3:1 or 1:2 to 2:1. It can furthermore be adequate in some cases, when the amount of constituent with the highest viscosity is about equal to or higher than the amount of constituent with the lowest viscosity, resulting in a value of from 0.9:1 to 3:1 for the ratio of the amount of constituent with the highest viscosity to the amount of constituent with the lowest viscosity. All of the ratios are based on the weight of the constituents.

According to another embodiment component (A) can be a QM resin containing vinyl groups.

QM resins comprise as Q a quadrifunctional SiO_{4/2} unit and as M building blocks such as monofunctional units R₃SiO_{1/2}, wherein R is vinyl, methyl, ethyl or phenyl or tri- or bi-functional units.

A preferred QM resin which can be used as component (A) has the structure: Si[O—Si(CH₃)₂—CH═CH₂]₄.

Examples of suitable QM resins are e.g. described in US 2005/0027032. The content of this document with respect to the description of QM resins is herewith incorporated by reference.

QM resins can be used in addition to the organopolysiloxanes described above or instead of the organopolysiloxanes described above.

A component (A) which can be employed can consist of one type (A1) of organopolysiloxane.

The organopolysiloxane can have a viscosity starting in the range of 1 to 1,000,000 mPas, or 5 to 500,000 mPas or 10 to 50,000 or 30 to 40,000 mPas (at 23° C.).

Thus, it is also possible that component (A) comprises two or more constituents, (A1), (A2) and so on, which can differ, e.g., in the chemical composition of their backbone, or their molecular weight, or their substituents or their viscosity, or any other differentiating feature or two or more of the above mentioned features.

According to another embodiment, curable component (A) is described by or comprises a component of the following formula (II):

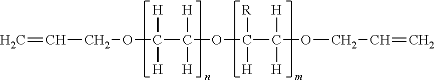

with n+m=3 to 300 or 10 to 200 and R being H or alkyl (e.g. C1 to C4).

If desired, this component may be used either alone or in combination with the organopolysiloxane according to formula (I) above.

Component (A) can be present in the following amounts:

Lower limit: at least 10 or at least 15 or at least 20 wt.-%;

Upper limit: utmost 60 or utmost 55 wt.-% or utmost 50 wt.-%;

Range: from 10 to 60 or from 15 to 55 wt.-% or from 20 to 50 wt.-%;

wt.-% with respect to the weight of the whole composition.

The hardenable composition described in the present text also comprises a crosslinker component as component (B) capable of crosslinking component (A).

The crosslinker compound is typically an organohydrogenpolysiloxane with at least 3 SiH groups per molecule.

An organohydrogenpolysiloxane for use as component (B) typically contains from about 0.01 to about 1.7 wt.-% silicon-bonded hydrogen or from 1.0 to 9.0 mmol SiH/g. The silicon valencies which are not saturated with hydrogen or oxygen atoms are typically saturated with monovalent hydrocarbon radicals R free from ethylenically unsaturated bonds.

The hydrocarbon radicals R, which may be selected independently from each other, represent a linear or branched or cyclic, non-substituted or substituted, aliphatic or aromatic monovalent hydrocarbon groups with 1 to 12 C atoms without ethylenically unsaturated bonds. In a preferred embodiment of the invention, at least 50%, preferably 100%, of the hydrocarbon radicals R that are bonded to silicon atoms are methyl radicals.

Organohydrogenpolysiloxanes which can be suitable as component (B) include those having a viscosity of 10 to 1,000 mPas or from 15 to 550 mPas or from 20 to 250 mPas (at 23° C.).

Component (B) can be present in the following amounts:

Lower limit: at least 0.1 or at least 1 or at least 3 wt.-%;

Upper limit: utmost 20 or utmost 15 or utmost 10 wt.-%;

Range: from 0.1 to 20 or from 1 to 15 or from 3 to 10 wt.-%;

wt.-% with respect to the weight of the whole composition.

The hardenable composition described in the present text comprises a catalyst as component (C) capable of catalyzing a crosslinking reaction of component (A) and component (B),

The nature of the catalyst to be used typically depends on the nature of the components (A) comprising curable moieties (AC) used.

If the hardenable composition cures according to an addition reaction, the catalyst is typically a platinum catalyst or a platinum containing catalyst, including a platinum complex which can be prepared from hexachloroplatinum acid by reduction with tetramethyldivinyldisiloxane. Such compounds are known to the skilled person.

Any other compounds which catalyze or accelerate addition cross-linking of silanes with ethylenically unsaturated double bonds are also suitable. Platinum-siloxane complexes as described, e.g. in U.S. Pat. Nos. 3,715,334, 3,775,352 and 3,814,730 are suitable. The disclosure of these patents with regard to platinum complexes and their preparation is explicitly mentioned and expressly regarded as part of the disclosure of the present text.

If the catalyst is a Pt containing catalyst, the catalyst component (C) may be present in the following amounts:

Lower amount: at least 0.00005 or at least 0.0002 wt.-%;

Upper amount: utmost 0.05 or utmost 0.04 wt.-%;

Range: from 0.00005 to 0.05 or from 0.0002 to 0.04 wt.-%,

calculated as elemental platinum and related to the overall weight of the composition.

The hardenable composition described in the present text comprises polymeric particles as filler component (D).

The polymeric particles are composed of organic polymers, silicone elastomers or a combination or mixture thereof.

The maximum particle size of the polymeric particles is 150 μm or below, or 140 μm below.

The particle size distribution of the polymeric particles may be characterized as follows: d50/μm=below 50 or d90/μm=below 100. D50/μm means that 50 percent of the analyzed particles have a diameter below 50 μm. D90/μm means that 90 percent of the analyzed particles have a diameter below 100 μm.

It was found that using polymeric particles having a larger particle size, in particular above 150 μm are not suitable for achieving the desired properties.

In addition the polymer particles should have a suitable hardness to allow a finely dispersion of the particles in the matrix.

Hardness often depends on a variety of properties including elasticity, viscosity, plasticity, toughness. If the hardness of the particles is too low, the particles might fibrillate during e.g. a kneading process and therefore may not improve the elastomeric matrix in the desired way. Those materials typically show a rubber like consistence. However, if the hardness of the particles is too high, there may be no significant improvement of the viscoelastic properties of the matrix.

Therefore, polymeric particles having a Shore A hardness below 70 or below 60 are often preferred.

The component(s) the polymeric particles are made are typically characterized by either or more or all of the following features:

being a silicone elastomer comprising dimethylsiloxane moieties and/or being a fluoropolymer characterized by a melting point below 335° C.;

being a fluoropolymer comprising more than 99% monomer repeating units of tetra fluoroethylene;

showing elastomeric properties like blockcopolymers of soft and hard segments e.g. like blockcopolymers of styrene, butadiene, acrylates, methycrylates or olefines.

Filler component (D) may also be characterized by at least one or more or all of the following features:

maximum particle size: 150 μm or below; or 140 μm or below;

d50/μm: 50 or below; or 40 or below; or 30 or below;

d90/μm: 100 or below; or 90 or below; or 80 or below;

average particle size: 1 to 30 or 1 to 25 or 1 to 20 μm;

BET surface according to DIN ISO 9277:1 to 20 m²/g;

bulk density according to DIN EN ISO 60: below 2 g/cm³ or from 0.1 to 2 g/cm³;

melting peak temperature according to DIN EN ISO 12086: above 100 or above 250° C. or from above 100° C. to below 340° C.;

molecular weight: 10⁴ to 10⁸ g/mol or 10⁵ to 10⁷ g/mol;

Shore hardness A: below 70 or below 60;

polymerization type: particles obtained by emulsion or suspension polymerization or thermal degradation;

non-swellable;

shape: essentially spherical.

Suitable polymeric filler particles are those which can be combined with the other components of the hardenable composition to obtain a paste. Thus, the polymeric filler particles should not dissolve in the hardenable composition.

According to one embodiment, filler component (D) comprises fluorine containing particles having either or all of the following properties:

maximum particle size: 150 μm or below; or 140 μm or below;

Shore hardness A: 70 or below; or 65 or below;

melting peak temperature: below 340° C. or below 335° C.

This kind of filler was found to be particularly useful for the desired purpose. Examples of filler component (D) comprising fluorine containing particles include: polytetrafluorethylene (PTFE) powder, tetrafluorethylene/hexafluorpropylene copolymer (FEP) powder, ethylene/tetrafluorethylene copolymer (ETFE) powder, tetrafluorethylene/hexafluorpropylene/vinylidenfluoride copolymer (THV) powder and mixtures thereof.

It was found that particles obtained from fully or per-fluorinated polymers are sometimes more suitable than particles obtained from partially fluorinated polymers.

A content of the tetra fluoro ethylene units (TFE) of the polymer forming the fluorine containing particles of above 95% or above 99% was found to be particular useful.

Further, it was found that polymeric particles obtained by emulsion polymerization are sometimes more suitable than polymeric particles obtained by suspension polymerization.

Specific examples for fluorine containing particles include the commercially available powders:

Dyneon: TF9201Z, TF9202Z, TF9207Z, TF9205,

Dupont: Zonyl™ MP1000, MP1100, MP1200.

According to another embodiment, filler component (D) comprises siloxane moieties containing particles.

According to one embodiment, the particles of the silicone elastomer are characterized as follows:

being a silicone elastomer comprising dimethylsiloxane moieties;

maximum particle size: 150 μm below; or 140 μm or below.

A specific example for siloxane moieties containing particles includes the commercially available silicone powder Cosmetic Powder 9701 (Dow Corning).

The polymeric particulate filler component (D) may be present in the following amounts:

Lower amount: at least 2 or at least 4 or at least 6 wt.-%;

Upper amount: utmost 70 or utmost 60 or utmost 50 wt.-%;

Range: from 2 to 70 or from 4 to 60 or from 5 to 50 wt.-%;

wt.-% with respect to the weight of the whole composition.

If the amount of the polymeric filler component (D) is too high, recovery after elongation might not be sufficient for dental impressioning purposes.

If the amount of the polymeric filler component (D) is too low, the desired effect of softening the rubber while not changing the paste consistency might not occur.

The hardenable composition described in the present text may also comprise an inorganic filler as filler component (E1).

Inorganic filler (E1) may be described by the following feature: having a BET surface of at least 50 m²/g, e.g. from 50 to 400 m²/g.

If desired, the BET surface of the filler can be determined as described in DIN 66132.

Suitable inorganic filler components (E1) include pyrogenic or fumed or precipitated silicic acid. Those filler are commercially available from companies like Wacker or Degussa under the trade names Aerosil™, HDK-H™ or Aeroxide™.

Those filler(s) are sometimes also referred to as reinforcing filler(s).

The surface of the filler component (E1) may be surface treated, e.g. by treatment with organosilanes or siloxanes or by the etherification of hydroxyl groups to alkoxy groups.

The surface treatment can be carried out, e.g. with dimethyldichlorosilane, hexamethyldisilazane, tetramethylcyclotetrasiloxane or polymethylsiloxane.

If present, the inorganic filler component (E1) may be present in the following amounts:

Lower amount: at least 0.1 or at least 0.5 or at least 1 wt.-%;

Upper amount: utmost 50 or utmost 20 or utmost 10 wt.-%;

Range: from 0.1 to 50 or from 0.5 to 20 or from 1 to 10 wt.-%;

wt-% with respect to the weight of the whole composition.

If the amount of the inorganic filler component (E1) is too high, the consistency and rheological properties of the pastes will not be adequate for an impression material to capture good detail accuracy of the preparation margins and teeth in a patient's mouth.

If the amount of the inorganic filler component (E1) is too low, the strengthening effect maybe too low and separation effects in the paste may occur, e.g. sedimentation of fillers.

If filler component (E1) is present, the ratio of polymeric filler component (D) to inorganic filler component (E1) in the whole composition is typically greater than 1 or greater than 2 or greater than 3 or greater than 4 with respect to weight.

The hardenable composition described in the present text may also comprise an inorganic filler as filler component (E2), filler component (E2) being different from filler component (E1) with respect to its mean primary particle size.

Inorganic filler (E2) may be described by the following features:

maximum particles size: 200 μm or below; or 150 μm or below;

BET surface: below 50 m²/g; or from 1 to 40 m²/g.

Examples for the inorganic filler component (E2) include quartz, cristobalite, calcium silicate, diatomaceous earth, zirconium silicate, montmorillonite such as bentonite, zeolite, including molecular sieves such as sodium aluminium silicate, metal oxide powder such as aluminium oxide, titanium oxide or zinc oxide or their mixed oxides, barium sulphate, calcium carbonate, plaster, glass and mixtures thereof.

If desired, the inorganic filler component (E2) can be surface-treated, as well. The surface treatment can generally be carried out with the same methods as described in the case of inorganic filler component (E1).

Those filler(s) are sometimes also referred to as non-reinforcing filler(s).

If present, the inorganic filler component (E2) may be present in the following amounts:

Lower amount: at least 0.1 or at least 1 or at least 10 wt.-%;

Upper amount: utmost 80 or utmost 75 or utmost 70 wt.-%;

Range: from 0.1 to 80 or from 1 to 75 or from 10 to 70 wt.-%;

wt.-% with respect to the weight of the whole composition.

If the amount of the inorganic filler component (E2) is too high, hardness of the set impression will become too high so that it can become difficult to remove it from a patient's mouth.

If the amount of the inorganic filler component (E2) is too low, the required hardness of the set impression to be able to pour it with gypsum without distortions may not be achieved.

Using a combination of reinforcing and non-reinforcing fillers can be preferred. In this respect, the quantity of reinforcing fillers can range from 0.1 to 10 wt.-%, in particular from 0.4 to 8 wt.-% with respect to the whole composition.

The hardenable composition described in the present text may also contain in addition additive component(s) (F).

Those additives include hydrophilating agent(s), retarder(s) to modify the working and setting time (e.g. 3-methyl-1-butyne-3-ol or 1,1,3,3-tetramethyl-1,3-divinyl siloxane (VMO)), inhibitor(s), colourant(s) (i.e. pigment(s) and dye(s)), stabilizer(s), plastizer(s) (including paraffin oil or mineral oil), flavouring(s), hydrogen scavenger(s), rheology modifier(s) (e.g. synthetic or natural waxes or polyethylene/propylene diacetats as described in EP 1 165 016 A1; corresponding to U.S. Pat. No. 6,677,393) etc. alone or in admixture.

To control the reactivity of the addition reaction and to prevent premature curing, it may be advantageous to add an inhibitor, which prevents the addition reaction for a specific period of time or slows the addition reaction down. Such inhibitors are known and described, e.g. in U.S. Pat. No. 3,933,880. This content of this reference regarding such inhibitors and their preparation is expressly regarded as being part of the disclosure of the invention and herewith incorporated by reference.

Examples of such inhibitors include acetylenic unsaturated alcohols such as 3-methyl-1-butyne-3-ol , 1-ethynylcyclohexane-1-ol, 3,5-dimethyl-1-hexyne-3-ol and 3-methyl-1-pentyne-3-ol. Examples of inhibitors based on vinyl siloxane are 1,1,3,3-tetramethyl-1,3-divinyl siloxane, 1,3,5,7-tetravinyl-1,3,5,7-tetramethylcyclotetrasiloxane and poly-, oligo- and disiloxanes containing vinyl groups.

The composition may also contain a component useful for diminishing the presence or degree of hydrogen outgassing which may be typically generated as a result of the vinyl polymerization in the case of SiH curable composition. The composition thus may comprise a hydrogen scavenger such as finely divided platinum metal that scavenges for and takes up such hydrogen. The Pt metal may be deposited upon a substantially insoluble salt having a surface area of between about 0.1 and about 40 m²/g. Suitable salts are Barium sulphate, barium carbonate and calcium carbonate of suitable particle sizes. Other substrates include diatomaceous earth, activated alumina, activated carbon and others. The inorganic salts are especially preferred to imply improved stability to the resulting materials incorporating them. Dispersed upon the salts is 0.2 to 2 parts per million of platinum metal, based upon the weight of the catalyst component. It has been found that employment of the platinum metal dispersed upon inorganic salt particles substantially eliminates or diminishes hydrogen outgassing during curing of dental silicones. Also Pd metal (e.g. as described e.g. in U.S. Pat. No. 4,273,902) or Pd compounds (e.g. as disclosed in to U.S. Pat. No. 5,684,060) can be employed.

The dental impression material may further contain a stabilizer as component, e.g. selected from antioxidants and mixtures thereof.

Useful antioxidant(s) which can be used include: Vitamin E; N,N′-di-2-butyl-1,4-phenylenediamine; N,N′-di-2-butyl-1,4-phenylenediamine; 2,6-di-tert-butyl-4-methylphenol; 2,4-dimethyl-6-tert-butylphenol; 2,4-dimethyl-6-tert-butylphenol and 2,6-di-tert-butyl-4-methylphenol; 2,6-di-tert-butylphenol; Pentaerythritoltetrakis(3-(3,5-di-tert-butyl-4-hydroxyphenyl)propionate) (Irganox™ 1010); Octyl-3,5-di-tert-butyl-4-hydroxy-hydrocinnamate; Octadecyl 3-(3,5-di-tert-butyl-4-hydroxyphenyl)propionate; 1,3,5-Trimethyl-2,4,6-tris(3,5-di-tert-butyl-4-hydroxybenzyl)benzene; 2,2,4,4-Tetrakis-tent-butyl-3,3-dihydroxybiphenyl; 4,4-Butylidenebis(6-tert-butyl-m-cresol); 4,4′-Isopropyliden-bis-(2-tert-butylphenol); 2,2′-methylenebis(6-nonyl-p-cresol); 1,3,5-tris(3,5-di-tert-butyl-4-hydroxybenzyl-)-1,3,5-triazine-2,4,6(1H,3H,5H)trione; or mixtures thereof. Particularly useful include antioxidants comprising a phenolic moiety, especially a sterically hindered phenolic moiety.

The hardenable composition may also contain at least one hydrophilizing agent.

Surfactants or hydrophilizing agents which can be employed can generally be chosen freely from all types of surfactants which improve the hydrophilicity of a silicone moiety containing material (especially, if curable via a hydrosilylation reaction).

Useful surfactants can generally be chosen from anionic, cationic or non-ionic surfactants or mixtures of two or more of such types of surfactants.

It can be preferred, if the hardenable composition comprises a non-ionic surfactant as a hydrophilizing agent or a mixture of two or more non-ionic surfactants.

Ethoxylated fatty alcohols can be used. Suitable examples are e.g. described in EP 0 480 238 B1.

Also preferred are non-ionic surface-active substances including nonylphenolethoxylates, polyethylene glycol-mono- and diesters, sorbitan esters and polyethylene glycol-mono- and diethers. Suitable examples are described e.g. in U.S. Pat. No. 4,782,101. The content of these documents with regard to hydrophilizing agents and their preparation is herewith incorporated by reference.

Suitable hydrophilizing agents also include wetting agents from the group of hydrophilic silicone oils, which are not capable of being covalently incorporated into the hardened polymer network. Suitable hydrophilizing agents are described e.g. in U.S. Pat. No. 4,657,959 and in EP 0 231 420 B1. The content of these documents with regard to hydrophilizing agents and their preparation is herewith incorporated by reference.

Suitable silicone moieties containing surfactants can be summarized under the following formula (III)

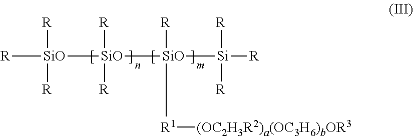

where each R is independently a monovalent hydrocarbyl radical with 1 to 22 C-atoms, R¹ is a divalent hydrocarbylene radical 1 to 26 C-atoms, each R² is independently hydrogen or a lower hydroxyalkyl radical, R³ is hydrogen or a monovalent hydrocarbyl radical with 1 to 22 C-atoms, n and b are independently greater than or equal to zero, and m and a are independently greater than or equal to one, with the proviso that a has a sufficient value and b is small enough so that a cured composition of the invention has the desired water contact angle.

Preferably R and R³ are —CH₃, R¹ is —C₃H₆—, R² is hydrogen, n is about zero or about one, m is about one to about five, a is about five to about 20 and b is about 0.

Several of such ethoxylated surfactants are for example available from Momentive Performance Materials Inc. including “Silwet™” surface active copolymers. Preferred surface active copolymers include Silwet 35, Silwet L-77, Silwet L-7600 and Silwet L-7602, Silwet L-7608 and Silwet Hydrostable 68 and Silwet Hydrostable 611. Silwet L-77 is an especially preferred ethoxylated surfactant which is believed to correspond to the above formula where R and R³ are —CH₃, R¹ is —C₃H₆—, R² is hydrogen, n is about zero or about one, m is about one or about two, a is about seven, and b is about 0. Also possible is the use of MASIL™ SF19, as obtainable from Lubrizol performance products, Spartanburg, US.

Useful surfactants also include polyether carbosilanes of the general formula Q-P—(OCₙH₂ₙ)ₓ—OT, in which Q stands for R₃—Si— or R₃—Si—(R′—SiR₂)ₐ—R′—SiR″₂— where every R in the molecule can be the same or different and stands for an aliphatic C₁-C₁₈, a cycloaliphatic C₆-C₁₂ or an aromatic C₆-C₁₂ hydrocarbon radical, which can optionally be substituted by halogen atoms, R′ is a C₁-C₁₄ alkylene group, R″ is R in the case of a≠0 or is R or R₃SiR′ in the case of a=0, and a=0-2; P stands for a C₂-C₁₈ alkylene group, preferably a C₂-C₁₄ alkylene group or A-R″′, where A represents a C₂-C₁₈ alkylene group and R″′ a functional group from the following list: —NHC(O)—, —NHC(O)—(CH2)ₙ₋₁-, —NHC(O)C(O)—, —NHC(O)(CH2)ᵥC(O)—, —OC(O)—, —OC(O)—(CH2)ₙ₋₁-, —OC(O)C(O)—, —OC(O)(CH₂)ᵥC(O)—, —OCH₂CH(OH)CH₂OC(O)(CH2)ₙ₋₁-, —OCH₂CH(OH)CH₂OC(O)(CH2)ᵥC(O)— with v=1-12; T is H or stands for a C₁-C₄ alkyl radical or a C₁-C₄ acyl radical; x stands for a number from 1 to 200 and n stands for an average number from 1 to 6, preferably 1 to 4. Thus, the element —SiR″₂— can also comprise the substructure —Si(R)(R₃SiR′)—.

The polyether part can be a homopolymer, but can also be a statistical, alternating or block copolymer.

Suitable polyether carbosilanes are selected from the group consisting of: Et₃Si—(CH₂)₃—O—(C₂H₄O)y—CH₃, Et=Ethyl; Et₃Si—CH₂—CH₂—O—(C₂H₄O)y—CH₃, Et=Ethyl; (Me₃Si—CH₂)₃Si—(CH₂)₃—O—(C₂H₄O)y—CH3, Me=Methyl; Me₃Si—CH₂—SiMe₂—(CH₂)₃—O—(C₂H₄O)y—CH₃, Me=Methyl; (Me₃Si—CH₂)₂SiMe—(CH₂)₃—O—(C₂H₄O)y—CH₃, Me=Methyl; Me₃Si—(CH₂)₃—O—(C₂H₄O)y—CH₃, Me=Methyl; Me₃Si—CH₂—CH₂—O—(C₂H₄O)y—CH₃, Me=Methyl; Ph₃Si—(CH₂)₃—O—(C₂H₄O)y—CH₃, Ph=phenyl; Ph₃Si—CH₂—CH₂—O—(C₂H₄O)y—CH₃, Ph=phenyl; Cy₃Si—(CH₂)₃—O—(C₂H₄O)y—CH₃, Cy=cyclohexyl; Cy₃Si—CH₂—CH₂—O—(C₂H₄O)y—CH₃, Cy=cyclohexyl; (C₆H₁₃)₃—(CH₂)₃—O—(C₂H₄O)y—CH₃, (C₆H₁₃)₃—Si—CH₂—CH₂—O—(C₄H₄O)y—CH₃ in which y conforms to the relation: 5≤y≤20 and mixtures thereof.

Surfactants which can also be used, either alone or as a mixture of two or more thereof, can be found in U.S. Pat. No. 5,750,589 (Zech et al), col. 2, 1. 47 to col. 3 1. 27 and col. 3, 1. 49 to col. 4, 1. 4 and col. 5, 1. 7 to col. 14, 1. 20.

Other surfactants which can be used, either alone or as a mixture of two or more thereof, can be found in U.S. Pat. No. 4,657,959 (Bryan et al.), col. 4, 1. 46 to col. 6. 1. 52 as well as in EP 0 231 420 B1 (Gribi et al.) p 4, 1. 1 to p. 5, 1. 16 and in the examples.

The content of these documents with regard to hydrophilizing agents and their preparation is herewith incorporated by reference.

In a particular embodiment, a mixture of a silicone moieties containing surfactant and one or more non-ionic surfactants selected from alkoxylated hydrocarbon surfactants is used.

Examples of useful non-ionic surfactants include those according to the formula:

R¹—O—[CH₂CH₂O]ₙ—[R²O]ₘ—R³

where R¹ represents an aromatic or aliphatic, linear or branched hydrocarbon group having at least 8 carbon atoms, R² represents an alkylene having 3 carbon atoms, R³ represents hydrogen or a C1-C3 alkyl group, n has a value of 0 to 40, m has a value of 0 to 40 and the sum of n+m being at least 2.

It will be understood that in the above formula, the units indexed by n and m may appear as blocks or they may be present in an alternating or random configuration. Examples of non-ionic surfactants according to the formula above include alkylphenol oxethylates such as ethoxylated p-isooctylphenol commercially available under the brand name TRITON™ such as for example TRITON™ X 100 wherein the number of ethoxy units is about 10 or TRITON™ X 114 wherein the number of ethoxy units is about 7 to 8. Still further examples include those in which R¹ in the above formula represents an alkyl group of 4 to 20 carbon atoms, m is 0 and R³ is hydrogen. An example thereof includes isotridecanol ethoxylated with about 8 ethoxy groups and which is commercially available as GENAPOL®X080 from Clariant GmbH. Non-ionic surfactants according to the above formula in which the hydrophilic part comprises a block-copolymer of ethoxy groups and propoxy groups may be used as well. Such non-ionic surfactants are commercially available from Clariant GmbH under the trade designation GENAPOL® PF 40 and GENAPOL® PF 80. Further suitable non-ionic surfactants that are commercially available include Tergitol™ TMN 6, Tergitol™ TMN 10, or Tergitol™ TMN 100X. Also statistical, alternating or block copolymers of ethylene oxide and propylene oxide are suitable surfactants according to the present invention. Such non-ionic surfactants are available e.g. under the trade name Breox™ A, Synperonic™ or Pluronic™.

Besides or in addition to the hydrophilazing agent(s) described above, the composition may comprise any of the following components:

ethylene oxide or propylene oxide polymers or ethylene-propylene block polymers bearing as end groups polymerizable moieties selected from vinly, allyl, —OCO—(CH₃)C═CH₂;

H₃—C—CO—[CH₂—CH₂—O—]ₘ—[CH₂—CH₂—CH₂—O—]ₙ—CO—CH₃ with n,m=10 to 100.

In addition to the hydrophilazing agent(s) mentioned above, the composition may also comprise an one or more F-containing component as hydrophilating agent.

Suitable examples of the F-containing compound include:

T₁-X—[(O—CF₂—CF₂)ₙ—(O—CF₂)ᵥ—(O—CF(CF₃)—CF₂)_{w}—(O—CF₂—CF₂—CF₂)ₓ—O]—X-T₂

with u=0 to 8, v=0 to 8, w=0 to 14 and x=0 to 8 and u+v+w+x≥1, wherein T₁ and T₂ can be equal or different and are independently selected from the group consisting of —COOR, —CONR^{b}R^{c}, —CH₂OH, —CF₂OR, —CHFOH, —CHFOR, —CH₂OR or —F with R and being a linear or branched alkyl rest (C1 to C9), aryl rest (C1 to C9) or alkylaryl rest (C1 to C9) each of which may optionally be substituted with one or more substituents selected from the group consisting of hydroxyl, amino group, halogen atom, an SiH group and a group capable of reacting with SiH, R^{b} and R^{c} independently representing H or having a meaning as given for R, and wherein X is selected from (CF₂)₁₋₆, CF(CF₃) and CHF—CF₂.

More precisely, the F-containing component can also be characterized by any of the following formulas:

Rf—(O)ₜ—CHF—(CF₂)ₓ-T, with t=0 or 1, x=0 or 1 and Rf being a linear or branched per- or partly fluorinated alkyl rest (including C1 to C6 or C1 to C4), wherein the alkyl chain can be interrupted by O atoms, with the proviso that when t is 0, the Rf group is a linear or branched per- or partly fluorinated alkyl rest (including C1 to C6 or C1 to C4) interrupted by one or more O atoms,

Rf—(OCF₂)ₘ—O—CF₂-T, with m=1 to about 6 and Rf being a linear or branched per- or partly fluorinated alkyl rest (including C1 to C6 or C1 to C4), wherein the alkyl chain can be interrupted by O atoms,

CF₃—(CF₂)₂—(OCF(CF₃)—CF₂)_{z}—O-L-T, with z=0, 1, 2, 3, 4, 5, 6, 7 or 8, L having a structure selected from —CF(CF₃)—, —CF₂—, —CF₂CF₂— and —CHFCF₂,

Rf—(O—CF₂CF₂)ₙ—O—CF₂-T, with n=1, 2, 3, 4 or 5 and Rf being a linear or branched per- or partly fluorinated alkyl rest (including C1 to C6 or C1 to C4), wherein the alkyl chain can be interrupted by O atoms,

an oligomeric compound obtainable by the anionic or photochemical (in the presence of oxygen) polymerization or copolymerization of monomers selected from vinylidenfluoride, hexafluoropropylenoxide, tetrafluoroethylene, 2,2,3,3-tetrafluorooxetane, trifluoroethylene or monofluoroethylene, wherein at least one chain-end of the oligomeric compound is represented by a function T,

T being selected from the group consisting of —COOR, —CONR^{b}R^{c}, —CH₂OH, —CF₂OR, —CHFOH, —CHFOR, —CH₂OR or —F with R and being a linear or branched alkyl rest (C1 to C9), aryl rest (C1 to C9) or alkylaryl rest (C1 to C9) each of which may optionally be substituted with one or more substituents selected from the group consisting of hydroxyl, amino group, halogen atom, an SiH group and a group capable of reacting with SiH, R^{b} and R^{c} independently representing H or having a meaning as given for R.

Specific examples of T include:

a) homo- or copolymerization of hexafluoropropylenoxide and/or 2,2,3,3-tetrafluorooxetane;

b) homo- or copolymerization of vinylidenfluoride, hexafluoropropylenoxide, tetrafluoroethylene, 2,2,3,3-tetrafluorooxetane, trifluoroethylene and/or monofluoroethylene in the presence of oxygen

In particular, the esters, especially the methylesters, and the amidols (T=C(O)NH-alkyl-OH) and the respective alcohols or methylethers, prepared by chemical reduction, of the following structures can be used.

Further examples can be found in EP 2 231 102 B1. The content of this reference with respect to the description of F-containing components is herewith incorporated by reference.

The F-containing components described above typically function as wetting-enabler, that is, they do not show hydrophiliating properties if used alone (i.e. without an additional surfactant), but increase the hydrophilating properties of an additionally added surfactant.

Particularly useful are hexafluoropropylene oxide (HFPO) derivatives including carboxyl ester derivatives and amidol derivatives of HFPO.

HFPO can be obtained as described in U.S. Pat. No. 3,242,218 or US 2004/0124396. The general formula of a methyl ester derivative of HFPO is C₃F₇O[CF(CF₃)CF₂O]ₙCF(CF₃)COOCH₃ with n being 2 to 14.

Besides curable components (A), the curable composition may also contain organopolysiloxanes without reactive substituents. Non-reactive substituents include those which do not co-polymerize with the other components of the composition during the hardening process. These are preferably linear, branched or cyclic organopolysiloxanes where all silicon atoms are surrounded by oxygen atoms or monovalent hydrocarbon radicals with 1 to 18 carbon atoms which can be substituted or non-substituted. The hydrocarbon radicals can be methyl, ethyl, C₂-C₁₀ aliphatics, trifluoropropyl groups as well as aromatic C₆-C₁₂ radicals.

Polydimethylsiloxanes with trimethylsiloxy end groups can be preferred and can be used in an amount of 0 to 40 wt.-%, or 0.1 to 20 wt.-% or 0.5 to 10 wt.-% with respect to the whole composition.

Astringent(s) which may be present include aluminium salts like aluminium sulfate, aluminium ammonium sulfated, aluminium chlorohydrated, aluminium acetate and mixtures thereof. Useful astringent(s) can also contain iron, manganese and/or zink containing substances.

The hardenable composition may comprise a flavorant or mixtures of flavorants to improve the taste and/or smell of the composition. Flavorants, which can be used, include isoamylacetate (banana), benzaldehyde (bitter almond), cinnamic aldehyde (Cinnamon), ethylpropionate (fruity), methyl anthranilate (Grape), mints (e.g. peppermints), limonene (e.g. Orange), Allylhexanoate (pineapple), ethylmaltol (candy), ethylvanillin (Vanilla), methyl salicylate (Wintergreen).

Examples of colourants which can be used include chinoline yellow dye (sicovit), chromophthalblue A3R, red iron oxide 3395, Bayferrox 920 Z Yellow, Neazopon Blue 807 (copper phthalocyanine-based dye), Helio Fast Yellow ER, Brilliant Blue FCF, Fast Green FCF and/or Orange Yellow S. Pigments or dyes which are stable under acidic conditions are preferred.

The use of colourants is sometimes preferred, as it may facilitate the visibility of the composition in the mouth of the patient, in particular, if the composition is used for dental retraction purposes.

If present, the additive component(s) (F) may be present in the following amounts:

Lower amount: at least 0.01 or at least 0.1 or at least 1 wt.-%;

Upper amount: utmost 20 or utmost 15 or utmost 10 wt.-%;

Range: from 0.01 to 20 or from 0.1 to 15 or from 1 to 10 wt.-%.

The hardenable composition described in the present text may contain the respective components in the following amounts:

Component (A): from 10 to 60 wt.-% or from 12.5 to 55 wt.-% or from 15 to 50 wt.-%,

Component (B): from 0.1 to 20 wt.-% or from 1 to 15 wt.-%,

Component (C) containing Pt: from 0.00005 to 0.05 wt.-% or from 0.00007 to 0.045 wt.-% or from 0.0001 to 0.04 wt.-%, calculated based on the Pt content,

Component (D): from 2.5 to 70 wt.-% or from 5 to 65 wt.-% or from 7.5 to 60 wt.-%,

Component (E1): from 0 to 20 wt.-% or from 0.1 to 20 wt.-% or from 0.1 to 15 wt.-% or from 1 to 10 wt.-%,

Component (E2): from 0 to 70 wt.-% or from 5 to 65 wt.-% or from 10 to 60 wt.-%,

Component (F): from 0 to 30 wt.-% or from 0.5 to 25 wt.-% or from 1 to 20 wt.-%,

wt.-% with respect to the weight of the whole composition.

The hardenable composition described in the present text may be provided as a kit of parts comprising a base part and a catalyst part which are kept separate from each other until use.

The base part typically comprises Component (A) and Component (B). The catalyst part typically comprises Component (C) and Component (A).

The other component(s) e.g. filler component (D1), filler component (E), optional filler component (D2), and the optional additive component(s) (F) may be present either in the base part only, or the catalyst part only or the base part and the catalyst part.

According to one embodiment, polymeric filler component (E) is present in the base part and the catalyst part.

According to one embodiment, the invention is directed to a kit of parts comprising a base part and a catalyst part kept separate from each other until use,

the base part comprising Component (A) and Component (B),

the catalyst part comprising Component (C) and Component (A),

component (D), optional component (E1) and (E2) and (F) being present either in the base part only, or the catalyst part only or the base part and the catalyst part, Component (A) comprising an organopolysiloxane with at least 2 aliphatically unsaturated groups and being present in an amount from 10 to 60 wt.-%,

Component (B) comprising an organohydrogenpolysiloxane with at least 3 SiH groups per molecule and being present in an amount from 0.1 to 20 wt.-%,

Component (C) being a Pt containing catalyst,

Component (D) comprising the polymeric particles in an amount from 2 to 70 wt.-%,

Component (E1) comprising fumed or precipitate silica in an amount from 0 to 20 wt.-% or 0.1 to 20 wt.-%,

Component (E2) comprising crystalline silicone dioxide in an amount from 0 to 70 wt.-%,

wt.-% with respect to the total amount of the composition.,

the hardenable composition being used for dental retraction or dental impression purposes.

The hardenable composition can be produced by mixing its respective components.

Suitable mixing devices include kneaders and speed mixers.

According to a further embodiment the invention is directed to a hardenable dental composition comprising:

(A) a curable component comprising curable moieties (AC), the curable component being an organopolysiloxane with at least 2 aliphatically unsaturated groups,

(B) a crosslinker component as component (B) capable of crosslinking said component (A), crosslinker component (B) being an organohydrogenpolysiloxane,

(C) a catalyst as component (C) capable of catalyzing a crosslinking reaction of component (A) and component (B), catalyst component being a Pt containing catalyst

(D) polymeric particles as filler component (D), the polymeric particles having a maximum particle size of 150 μm or below and being a fluoropolymer as described in the present text, the fluoropolymer having typically a melting point below 335° C. and/or being typically a fluoropolymer comprising more than 99% monomer repeating units of tetra fluoroethylene.

According to a further embodiment the invention is directed to a hardenable dental composition comprising:

(A) a curable component comprising curable moieties (AC), the curable component being an organopolysiloxane with at least 2 aliphatically unsaturated groups,

(B) a crosslinker component as component (B) capable of crosslinking said component (A), crosslinker component (B) being an organohydrogenpolysiloxane,

(C) a catalyst as component (C) capable of catalyzing a crosslinking reaction of component (A) and component (B), catalyst component being a Pt containing catalyst

(D) polymeric particles as filler component (D), the polymeric particles having a maximum particle size of 150 μm or below and being a silicone elastomer as described in the present text, the silicone elastomer comprising dimethylsiloxane moieties.

If the hardenable composition described in the present text is provided as a kit of parts, the base part and the catalyst part are typically contained in separate compartments of a container.

The compartments typically comprise a front end and a rear end, wherein the front end of each compartment has an outlet opening, which can be connected to a mixing cannula, and wherein the rear end comprises a moveable plunger.

The volume ratios of catalyst part and base part can range from about 10:1 to about 1:10. Particularly preferred volume ratios of base part to catalyst part are about 1:1 and about 5:1 (5 parts of base part to 1 part of catalyst part).

Generally, mixing and dosing of the individual parts or pastes can be performed manually, e.g., by spatula (strand-length comparison) or a manually operated pre-filled dual cartridge dispenser with static mixing tips, or automated, using one of the various available devices available for such an automated task, preferably one of the devices mentioned in EP 0 232 733 A1, U.S. Pat. Nos. 5,924,600, 6,135,631 or EP 0 863 088 A1 together with a dynamic mixing tip as mentioned in US 2004/0085854 or U.S. Pat. No. 6,244,740.

A further improvement of the handling properties of hardenable dental compositions can be seen in using an automatic mixing and metering systems for two-component compositions which have automatic conveying and mixing units, such as are described e.g. in U.S. Pat. Nos. 5,249,862, 5,286,105 and 5,332,122. The need for manual mixing of base pastes and catalyst pastes, above all when mixing larger quantities of material, can be eliminated, since this can take place automatically and within a short period of time. The result is usually a homogeneous product which is essentially free of air bubbles. Commercially available devices are distributed by 3M ESPE under the brand Pentamix™ or Pentamix™ 2 or Pentamix™ 3. Certain embodiments of the hardenable composition described in the present text are suitable to be mixed by such a system and are thus called “auto-mixer suitable”.

In practice, the hardenable dental composition can also be syringed through a static or mechanical mixing device into an impression tray or onto patient's teeth or tissue and placed in the patient's mouth. After the material is set, the tray is removed from the patient's mouth and, in instances where the dental practitioner prepares the positive model, it may be preferable to pour the positive model material after removal of the impression from the patient's mouth with gypsum.

The invention is also directed to the use of the polymeric particles described as component (D) in the present text for producing a hardenable dental composition as described in the present text.

The obtained hardenable dental composition is in particular useful as dental impression material or for conducting a dental impression process.

The dental impression materials can be used as precision impression materials, situation impression materials or bite registration impression materials.

The dental impression material can also be used for the production of (temporary or long term) crown and/or bridges. In the latter case, the composition is used as a mould to be filled with the (temporary or long term) crown and/or bridge material, which is typically based on polymerizable (meth)acrylates.

In this respect the invention is directed to a process of taking an impression of the dental situation in the mouth of a patient, the process comprising the steps of

providing the hardenable composition as described in the present text,

bringing the hardenable composition in contact with the surface of the dental situation in the mouth of a patient,

letting the hardenable composition cure,

removing the hardened composition from the mouth of the patient.

The hardenable composition described in the present text can also be used as dental retraction material or for the purpose of retracting soft dental tissue from hard dental tissue.

Due to the presence of polymeric particles as filler component in the hardenable composition, the viscosity and consistency of the obtained composition can be adjusted such that the hardenable composition can not only be easily placed in the sulcus of a teeth but also exerts sufficient pressure on the surrounding soft tissue having the result that the sulcus is widened. Due to its elastomeric properties in its cured stage, the composition can also be easily removed from the sulcus after hardening.

Thus, the curable dental composition described in the present text is not only suitable as dental impression material but also as dental retraction material.

The invention is also directed to the use of the polymeric particles as described in the present text for lowering the Shore hardness and increasing the elongation at break value of the hardened composition while maintaining the consistency of the hardenable dental composition described in the present text.

The hardenable composition described in the present text is in particular to be used in the dental or orthodontic area. That is, the hardenable composition does typically not contain or produce components during hardening which may be detrimental to the patient's health.

Examples of components which are typically not comprised are either, more or all of the following:

silicone oil having a viscosity above 2,000,000 mPa*s at 23° C.,

halogenated solvents,

cellulose and or starch,

salts of polyacrylic acids and/or

fibres.

Further, the composition described in the present text does not contain polymeric particles having a particle diameter of more than 150 μm in an amount of more than 2 wt.-% or more than 1 wt.-% or more than 0.5 wt.-% with respect to the weight of the whole composition.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Measurements

### Shore Hardness A

The Shore hardness A was determined according to DIN 53505:2000-08

### Consistency (Cons)

The consistency was determined according to ISO 4823:2000-12.

### Elongation at Break (Elongation)

The elongation at break was determined according to DIN 53504:2009-10.

### Maximum Particle Size and Particle Size Distribution

The maximum particle size can be determined by laser diffraction using a Cilas 1064 Granulometer in “Dry Mode”. Results are calculated using the Fraunhofer approximation without Mie correction.

### Viscosity

If desired, the viscosity can be measured at 23° C. using a Haake Rotovisco™ 1 device with a plate/plate system (diameter 20 mm) and a slit of 0.2 mm. The viscosity values (Pas) and share stress values (Pa) can be recorded for each share rate (starting from 10 l/s to 110 l/s in 10 l/s steps. For each share rate, a delay of 5 s was used before collecting data. The above mentioned method of measurement corresponds essentially to DIN 53018-1.

### Water Contact Angle

If desired, the water contact angle of the uncured paste can be measured as follows: Test specimen preparation: For the preparation of test piece the mixed paste is subjected to an object slide and flattened and triturated by a second object slide in order to obtain a thin film. The test piece preparation is performed in that simplified way as the thickness of the film does not have a significant effect on the measured water contact angle (see G. Kugel, T. Klettke, J. A. Goldberg, J. Benchimol, R. D. Perry, S. Sharma, J. Prosthod. 2007, 16, 84-92). Measurement: The object slide is placed on the table of a Drop Shape Analyse System DSA 10 (Krüss GmbH, Hamburg), a well known device for measuring contact angles. 5 μl of water are placed onto the surface of the specimen and an automatic contact angle measurement is started using standard software of the goniometer. Measuring time is at least about 10 s up to about 200 s. The water contact angle is measured at different time periods after mixing of base paste and catalyst paste, especially after 25 s. The data (video sequences) is evaluated by the “circle fitting” method, another standard method for data evaluation (see G. Kugel, T. Klettke, J. A. Goldberg, J. Benchimol, R. D. Perry, S. Sharma, J. Prosthod. 2007, 16, 84-92); Θ 2 s is the angle obtained 2 s after placing the water drop on the surface.

### Swellability

If desired, the swellability can be determined as follows: 5 g material or particles are dispersed in 50 ml water (23° C.) and stirred for 5 min. The suspension is filtrated. The material or particles are put in water and the volume expansion of the material or particles determined with the Archimedes method (i.e. by displacement of water).

### Stickiness

The composition was kneaded by hand using gloves. The composition was classified as “non tacky” if the material does not stick to gloves (made of nitrile rubber).

### Materials

The maximum particle size of the polymeric powders were determined according to the description given above.

| | | |
|---|---|---|
| Vinyl terminated | CAS: 68093-19-2 | |
| Polydimethylsiloxane | | |
| Polymethylhydrogensiloxane | CAS: 68037-59-2 | |
| Polyether surfactant | Silwet ™ L 77 | |
| Desmosint ™ X92A-1 | Polyurethane powder | max. particle size: 180 μm |
| | (Lehmann&Voss) | |
| TF 9207Z | PTFE particles (Dyneon) | max. particle size: 46 μm |
| TF 1620 | PTFE particles (Dyneon) | max. particle size: 300 μm |
| Zonyl ™ MP 1000 | PTFE particles (Dupont) | max. particle size: 110 μm |
| Cosmetic Powder 9701 | Silicone powder (Dow Chemical) | max. particle size: 140 μm |
| Imprint ™ 4 Regular | VPS based dental impression | |
| | material (3M ESPE) | |
| Express ™ STD | VPS based dental impression | |
| | material (3M ESPE) | |
| Identium ™ medium | Vinylsiloxanether based | |
| | Impression material (Kettenbach) | |

### Example A—General Procedure

A Base Paste (B) and a Catalyst Paste (C) were provided as described below. To each paste a certain amount of inorganic filler and/or polymeric filler was added and mixed with a speed mixer to provide a homogeneous mixture. The Base Paste (B) and Catalyst Paste (C) were mixed in a ratio of 1:1 with respect to volume. The resulting composition/mixture was examined with respect to consistency, Shore hardness and elongation at break.

| Base Paste (B) | Amount [g] |
|---|---|
| Polymethylhydrogensiloxane (4.00 mmol/g SiH, 100 mPas) | 7.000 |
| Polymethylhydrogensiloxane (1.78 mmol/g SiH, 50 mPas) | 6.000 |
| Vinyl terminated polydimethylsiloxane, 200 mPas | 19.000 |
| Vinyl terminated polydimethylsiloxane, 7000 mPas | 24.750 |
| Polydimethylsiloxane, 10 mPas | 7.750 |
| Hydrophobized fumed silica (100 m²/g) | 4.000 |
| Pigment | 0.500 |
| Polyether surfactant | 1.500 |

| Catalyst Paste (C) | Amount [g] |
|---|---|
| Vinyl terminated polydimethylsiloxane, 200 mPas | 13.700 |
| Vinyl terminated polydimethylsiloxane, 7000 mPas | 27.600 |
| Polydimethylsiloxane, 10 mPas | 4.000 |
| Hydrophobized fumed silica (100 m²/g) | 2.600 |
| Platinum catalyst solution | 1.600 |
| Tetraallylsilane | 0.500 |

| | Polymeric filler/ | | |
|---|---|---|---|
| | total amount of | Inorganic Filler | Polymeric Filler |
| Ex- | filler | B/C/M [g/100 g] | B/C/M [g/100 g] |
| ample | [%] | Cristobalite | TF 9207Z |
| I-E1 | 0 | 29.500/50.000/39.750 | 0.000/0.000/0.000 |
| I-E2 | 25 | 17.700/30.000/23.850 | 5.900/10.000/7.950 |
| I-E3 | 50 | 8.850/15.000/11.925 | 8.850/15.000/11.925 |
| I-E4 | 75 | 3.688/6.250/4.969 | 11.063/18.750/14.907 |
| I-E5 | 100 | 0.000/0.000/0.000 | 12.390/21.000/16.695 |
| | | | Zonyl ™ MP 1000 |
| I-E6 | | 0.000/0.000/0.000 | 14.95/29.55/22.26 |
| | | | TF 1620 |
| I-E7* | | 0.000/0.000/0.000 | 22.89/32.36/27.62 |
| | Consistency | | |
| | (Cons) | Shore A | Elongation |
| | M [mm] | 24 h | [%] |
| I-E1 | 46 | 60 | 212 |
| I-E2 | 47 | 52 | 220 |
| I-E3 | 48 | 47 | 213 |
| I-E4 | 48 | 43 | 250 |
| I-E5 | 48 | 40 | 275 |
| I-E6 | 43 | 40 | 305 |
| I- | No paste like | — | — |
| E7* | consistency | | |

M = mixture of B and C;
*Comparative Example

With increasing the content of the polymeric filler compared to the inorganic filler, the consistency of the respective pastes remained fairly constant, whereas the Shore hardness decreased. Simultaneously, an increase of the elongation value at break was observed. The usage of PTFE polymeric particles with a particle size outside the claimed range lead to an undesired non paste like consistency.

### Examples B/C

The commercially available products Imprint™ 4 Regular (3M ESPE; Lot #504369) or Identium™ medium (Kettenbach; Lot #130051-03) were modified by adding different amounts and types of polymeric particles to each base and catalyst paste. The polymeric particles were incorporated in the pastes by using a Speedmixer (Hausschild DAC 600.1 VAC-P). Mixing conditions: 30 s at 1700 rpm no vacuum; 60 s at 1700 rpm under vacuum; 240 s at 800 rpm under vacuum; 30 s at 2350 rpm under vacuum.

Base and catalyst paste were filled in a dual barrel cartridge (SulzerMixpac) equipped with a static mixing tip and mixed.

| | Additional amount | Cons | | |
|---|---|---|---|---|
| | of polymeric filler | M | Shore A | Elongation |
| | [%] | [mm] | 24 h | [%] |
| Imprint ™ 4 | | | | |
| Regular | | | | |
| I-E8 | 0 | 37 | 53 | 218 |
| I-E9 | TF 9207Z 10 | 32 | 54 | 242 |
| I-E10 | TF 9207Z 20 | 28.5 | 52 | 287 |
| I-E11 | TF 9207Z 30 | 23 | 52 | 261 |
| I-E12 | TF 9207Z 40 | 18 | 53 | 278 |
| I-E13* | Desmosint ™ | 26 | 63 | 142 |
| | X92A-1 40 | | | |
| Identium ™ medium | | | | |
| I-E14 | 0 | 36 | 61 | 178 |
| I-E15 | TF 9207Z 20 | 20 | 62 | 171 |

By adding polymeric filler with a particle size <150 μm the Shore A hardness remained fairly constant while the consistency increased. Simultaneously, an increase of the elongation value at break was observed for Imprint™ 4 Regular. Adding polymeric filler with a larger particle size, the consistency increased as well, but the Shore A hardness increased, too. The elongation decreased to an undesired level.

### Example D

The commercially available product Express™ STD (3M ESPE; Lot #N332018) has a Shore hardness A of 74 after 24 h and a consistency of 33 mm (base) and 33 mm (catalyst). Express™ STD contains limestone as filler.

The formulation of Express™ STD was manufactured in the lab in a vacuum-mixer in a scale of 350 g of base and catalyst paste. During the preparation of the material, in the base paste and the catalyst paste a part of the limestone filler of the formulation of Express™ STD was substituted.

### Catalyst Paste (B-C)

10 pbw (parts by weight) of limestone in the commercially available formulation of Express™ STD Putty catalyst paste were substituted by 7 parts by weight (pbw) of Cosmetic Powder 9701 (Silicone Powder from Dow Corning). Further, 2 pbw silicone oil with a high viscosity of 1,000,000 mPas (23° C.) were introduced.

### Base Paste (B-B)

10 pbw of limestone in the commercially available formulation of Express™ STD Putty were substituted by 7 pbw of Cosmetic Powder 9701 (Silicone Powder from Dow Corning). Further, 2 pbw silicone oil with a high viscosity of 1,000,000 mPas and 0.1 pbw of Silwet™ L77 surfactant were introduced.

Base paste B-B and Catalyst paste B-C were mixed and the obtained composition analysed.

Shore hardness A: 63 (determined after 24 h);

Consistency: 26 and 27 mm (according to ISO 4823);

Oil separation: no;

Stickiness: no.

The materials were thicker than the commercially available Express™ STD pastes. However, in its cured state the hardness of the product was decreased. The pastes looked dry and no oil separation was visible to the human eye. When mixed by hand the pastes were not sticky.

1. A hardenable dental composition comprising:
(A) curable component comprising curable moieties (AC),
(B) crosslinker component as component (B) capable of crosslinking said component (A),
(C) catalyst as component (C) capable of catalyzing a crosslinking reaction of component (A) and component (B),
(D) polymeric particles as filler component (D), the polymeric particles having a maximum particle size of 150 μm or below and being composed of organic polymers, silicone elastomers or mixture thereof,
(E1) an inorganic filler component having a BET surface from 50 to 400 m²/g.

2. The hardenable dental composition of claim 1, filler component (D) being characterized by at least one or more of the following features:
d50/μm: 50 or below;
d90/μm: 100 or below;
average particle size: 1 to 30 μm;
BET surface according to DIN ISO 9277: 1 to 20 m²/g;
Bulk density according to DIN EN ISO 60: below 2 g/cm³;
Melting peak temperature according to DIN EN ISO 12086: above 100° C.;
Shore hardness A: below 70 determined after 24 h according to DIN 53505;
Molecular weight: 10⁴ to 10⁸ g/mol;
Polymerization type: particles obtained by emulsion or suspension polymerization or thermal degradation,
Non-swellable.

3. The hardenable dental composition of claim 2, the component(s) the polymeric particles are made of being characterized by either of the following:
being a silicone elastomer comprising dimethylsiloxane moieties and/or being a fluoropolymer characterized by a melting point below 335° C.;
being a fluoropolymer comprising more than 99% monomer repeating units of tetra fluoroethylene;
showing elastomeric properties like blockcopolymers comprising soft and hard segments.

4. The hardenable dental composition of claim 3, filler component (D) being present in an amount of 2 to 70 wt.-% with respect to the weight of the whole composition.

5. The hardenable composition of claim 4, comprising in addition an inorganic filler component (E2), inorganic filler component (E2) being characterized by at least one or all of the following features:
maximum particle size: 200 μm or below;
BET surface: below 50 m²/g.

6. The hardenable dental composition of claim 1, being characterized by at least one or all of the following parameters after hardening:
Shore hardness A: from 35 to 65 determined after 24 h according to DIN 53505;
Elongation at break: at least 50% determined according to DIN 53504;
Curing time: composition reaches a Shore hardness A of at least 25 within 15 min at 23° C. after mixing;
Water contact angle: less than 20° or less than 13° at a water drop age of 10 s, 60 s after mixing.

7. The hardenable dental composition of claim 1, containing the following components in the following amounts:
Component (A): from 10 to 60 wt.-%,
Component (B): from 0.1 to 20 wt.-%,
Component (C) containing Pt: from 0.00005 to 0.05 wt.-% calculated on the Pt content,
Component (D): from 2 to 70 wt.-%,
Component (E1): from 0.1 to 20 wt.-%,
Component (E2): from 0 to 70 wt. %,
wt.-% with respect to the weight of the whole composition.

8. The hardenable dental composition of claim 1, provided as a kit of parts comprising a base part and a catalyst part kept separate from each other until use,
the base part comprising:
Component (A),
Component (B),
the catalyst part comprising:
Component (C) and
Component (A),
component (D), optional components (E1), (E2) being present either in the base part only, or the catalyst part only or the base part and the catalyst part, wherein Components (A), (B), (C), (D), and (E1).

9. The hardenable dental composition of claim 1, not comprising at least one or all of the following components:
silicone oil having a viscosity above 2,000,000 Pa*s at 23° C.;
salts of polyacrylic acid;
cellulose or starch powder;
fibres.

10. The hardenable dental composition of claim 1, provided as a kit of parts comprising a base part and a catalyst part kept separate from each other until use,
the base part comprising:
Component (A),
Component (B),
the catalyst part comprising:
Component (C) and
Component (A),
component (D), optional component (E1) and (F) being present either in the base part only, or the catalyst part only or the base part and the catalyst part,
Component (A) comprising an organopolysiloxane with at least 2 aliphatically unsaturated groups and being present in an amount from 10 to 60 wt.-%,
Component (B) comprising an organohydrogenpolysiloxane with at least 3 SiH groups per molecule and being present in an amount from 0.1 to 20 wt.-%,
Component (C) being a Pt containing catalyst,
Component (D) comprising the polymeric particles in an amount from 2 to 70 wt.-%,
Component (E1) comprising fumed or precipitate silica in an amount from 0.1 to 20 wt.-%,
wt.-% with respect to the total amount of the composition,
the hardenable composition to be used for dental retraction or dental impression purposes.

11. The hardenable dental composition of claim 10, the base part and catalyst part being contained in separate compartments of a container, the compartments comprising a front end and a rear end, the front end of each compartment having an outlet opening which can be connected to a mixing cannula, the rear end comprising a moveable plunger.

12. Use of a polymeric particulate filler component (D) for producing a hardenable dental composition of claim 1, the hardenable dental composition for use as dental impression or dental retraction material or for dental impression or dental retraction purposes.

13. Use of a polymeric particulate filler component (D) of claim 1 for lowering the Shore hardness and increasing the elongation at break value of the hardened dental composition while maintaining the consistency of the hardenable dental composition.

14. A process of taking a dental impression of the dental situation in the mouth of a patient or conducting a retraction, the process comprising the steps of:
providing the hardenable dental composition of claim 1,
bringing the hardenable dental composition in contact with the surface of the dental situation in the mouth of a patient,
letting the hardenable dental composition cure,
removing the hardened dental composition from the mouth of the patient.

15. The hardenable dental composition of claim 5, containing the following components in the following amounts:
Component (A): from 10 to 60 wt.-%,
Component (B): from 0.1 to 20 wt.-%,
Component (C) containing Pt: from 0.00005 to 0.05 wt.-% calculated on the Pt content,
Component (D): from 2 to 70 wt.-%,
Component (E1): from 0.1 to 20 wt.-%,
Component (E2): from 0 to 70 wt.-%,
wt.-% with respect to the weight of the whole composition.

16. The hardenable dental composition of claim 5, provided as a kit of parts comprising a base part and a catalyst part kept separate from each other until use,
the base part comprising:
Component (A),
Component (B),
the catalyst part comprising:
Component (C) and
Component (A),
component (D), optional components (E1), (E2) being present either in the base part only, or the catalyst part only or the base part and the catalyst part, wherein Components (A), (B), (C), (D), (E1) and (E2).

17. The hardenable dental composition of claim 5, provided as a kit of parts comprising a base part and a catalyst part kept separate from each other until use,
the base part comprising:
Component (A),
Component (B),
the catalyst part comprising:
Component (C) and
Component (A),
component (D), optional component (E1) and (E2) and (F) being present either in the base part only, or the catalyst part only or the base part and the catalyst part,
Component (A) comprising an organopolysiloxane with at least 2 aliphatically unsaturated groups and being present in an amount from 10 to 60 wt.-%,
Component (B) comprising an organohydrogenpolysiloxane with at least 3 SiH groups per molecule and being present in an amount from 0.1 to 20 wt.-%,
Component (C) being a Pt containing catalyst,
Component (D) comprising the polymeric particles in an amount from 2 to 70 wt.-%,
Component (E1) comprising fumed or precipitate silica in an amount from 0.1 to 20 wt.-%,
Component (E2) comprising crystalline silicone dioxide in an amount from 0 to 70 wt.-%,
wt.-% with respect to the total amount of the composition,
the hardenable composition to be used for dental retraction or dental impression purposes.
